(19) United States
(12) Patent Application Publication
(10) Pub. No.: US 2010/0197800 A1
(43) Pub. Date: Aug. 5, 2010

(54) Treatment and/or Prevention of Presbycusis by Modulation of Metabotropic Glutamate Receptor 7

(76) Inventors: Richard Friedman, Pacific Palisades, CA (US); Dietrich Stephan, Great Falls, VA (US); Guido (Guy) Van Camp, Duffel (BE); Matthew J. Huentelman, Phoenix, AZ (US); Lut Van Laer, Ghent (BE)

Correspondence Address: WILSON, SONSINI, GOODRICH & ROSATI, 650 PAGE MILL ROAD, PALO ALTO, CA, 94304-1050, US

(21) Appl. No.: 12/597,054

(22) PCT Filed: Apr. 23, 2008
(86) PCT No.: PCT/US08/61330

§ 371 Date: Mar. 19, 2010

Related U.S. Application Data
(60) Provisional application No. 60/913,520, filed on Apr. 23, 2007.
(60) Provisional application No. 61/004,880, filed on Nov. 30, 2007.
(60) Provisional application No. 61/021,007, filed on Jan. 14, 2008.

(51) Int. Cl. A61K 31/137; C07C 211/27; A61P 27/16

(52) U.S. Cl. 514/648; 564/372

(57) ABSTRACT

The invention relates to the treatment and/or prevention of age-related hearing loss (presbycusis) with a modulator of a metabotropic glutamate receptor 7 (mGluR7).

### FIELD OF THE INVENTION

The invention relates to the method of treatment and/or prevention of presbycusis by administering to a patient in need thereof a modulator of a metabotropic glutamate receptor 7 (mGluR7).

### DESCRIPTION OF THE RELATED ART

Presbycusis is the loss of hearing that gradually occurs in most individuals as they grow older. About 30-35 percent of adults between the ages of 65 and 75 years have a hearing loss. It is estimated that 40-50 percent of people 75 and older have a hearing loss.

The loss associated with presbycusis is usually greater for high-pitched sounds. For example, it may be difficult for someone to hear the nearby chirping of a bird or the ringing of a telephone. However, the same person may be able to hear clearly the low-pitched sound of a truck rumbling down the street.

There are many causes of presbycusis. Most commonly it arises from changes in the inner ear of a person as he or she ages, but presbycusis can also result from changes in the middle ear or from complex changes along the nerve pathways leading to the brain. Presbycusis most often occurs in both ears, affecting them equally. Because the process of loss is gradual, people who have presbycusis may not realize that their hearing is diminishing.

With presbycusis, sounds often seem less clear and lower in volume. This contributes to difficulty hearing and understanding speech. Individuals with presbycusis may experience several of the following: The speech of others seems mumbled or slurred. High-pitched sounds such as “s” and “th” are difficult to hear and tell apart. Conversations are difficult to understand, especially when there is background noise. A man's voice is easier to hear than the higher pitches of a woman's voice. Certain sounds seem annoying or overly loud. Tinnitus (a ringing, roaring, or hissing sound in one or both ears) may also occur.

### Types of Presbycusis

There are four different types of presbycusis: Sensory presbycusis results in abrupt loss of the ability to hear high frequencies and tones. Neural presbycusis reduces the ability to understand speech. Strial or metabolic presbycusis produces relatively flat hearing loss. Cochlear conductive presbycusis is characterized by a more gradual loss of the ability to hear high frequencies.

### Causes of Presbycusis

Sensorineural hearing loss is caused by disorders of the inner ear or auditory nerve. Presbycusis is usually a sensorineural hearing disorder. It is most commonly caused by gradual changes in the inner ear. The cumulative effects of repeated exposure to daily traffic sounds or construction work, noisy offices, equipment that produces noise, and loud music can cause sensorineural hearing loss. Sensorineural hearing loss is most often due to a loss of hair cells (sensory receptors in the inner ear). This can occur as a result of hereditary factors as well as aging, various health conditions, and side effects of some medicines (aspirin and certain antibiotics).

Presbycusis may be caused by changes in the blood supply to the ear because of heart disease, high blood pressure, vascular (pertaining to blood vessels) conditions caused by diabetes, or other circulatory problems. The loss may be mild, moderate, or severe.

Sometimes presbycusis is a conductive hearing disorder, meaning the loss of sound sensitivity is caused by abnormalities of the outer ear and/or middle ear. Such abnormalities may include reduced function of the tympanic membrane (the eardrum) or reduced function of the three tiny bones in the middle ear that carry sound waves from the tympanic membrane to the inner ear.

### Therapy Options

Currently, treatment options for presbycusis include treatment of postulated underlying causes, such as hypertension; hearing aids or a cochlear implant; assistive listening devices, such as telephone amplifiers; and removal of earwax. There are no clinically proven remedies for hearing loss, and a drug that could be used to prevent, alleviate or eliminate these symptoms would thus be very desirable from a clinical point of view.

### Glutamate Receptors

L-glutamate [L-Glu] is the primary excitory amino acid neurotransmitter in the mammalian central nervous system. It activates both ionotropic glutamate receptors (iGluRs) and metabotropic glutamate receptors (mGluRs). The former are coupled to ion-channels and typically mediate fast excitory neurotransmission. In this regard, NMDA (iGluR) receptor antagonists, such as Dizociipine (MK 801), have been proposed for the treatment of hearing loss. See e.g., US 2007/0015727; incorporated herein in its entirety by reference thereto. Unfortunately, such NMDA receptor antagonists may have serious psychotropic side effects.

In contrast to the iGluRs, the mGluRs are G-protein coupled receptors functioning via second messenger pathways to modulate neuronal excitability and synaptic efficacy. To date, eight subtypes of mGluRs have been identified, and they can be classified into three groups based on their sequence similarities, second messenger coupling and pharmacology. Group I (mGluR1 and mGluR5) couple to Gq, activate phospholipase C and are selectively activated by 3,5-dihydroxyphenyl glycine (DHPG) at low μM concentration. In contrast, Group II (mGluR2 and mGluR3) and Group 111 (mGluR4, 6, 7, 8) negatively couple via Gi/Go to adenylate cyclase and inhibit stimulated cAMP formation. Group II mGluRs can be selectively activated by (2S,1'S,2'S)-2-(dicarboxycyclopropyl)glycine (DCG-IV), whereas Group III mGluRs are selectively activated by synthetic agonist L-amino-4-phosphonobutyric acid (L-AP4) and endogenous ligand L-serine-O-phosphate (L-SOP).

There remains an important and unmet need to better understand the genetic basis and biology of presbycusis develop safer and more effective pharmacologic therapies for treating and/or preventing.

### SUMMARY OF THE INVENTION

In preferred embodiments, the invention relates to methods for preventing and/or treating presbycusis in a human. The methods include administering to a human a therapeutically effective amount of a pharmaceutical composition comprising an mGluR7 receptor modulator. In one embodiment, the modulator is a selective mGluR7 agonist. In another embodiment, the selective mGluR7 agonist is AMN082.

In one embodiment, the use of an mGluR7 modulator is disclosed for the preparation of a medicament for the treatment or prevention of age-related hearing loss (presbicusis). The modulator is preferably a selective mGluR7 agonist. More preferably, the selective mGluR7 agonist is AMN082. The medicament may be formulated for topical, oral or pump delivery, or via round or oval window delivery in accordance with an embodiment of the invention.

A pharmaceutical composition for treating or preventing age-related hearing loss is disclosed in accordance with an embodiment of the invention. The composition preferably comprises a modulator of mGluR7, such as the selective mGluR7 agonist, AMN082.

### BRIEF DESCRIPTION OF THE DRAWINGS

FIG. 1. Top 250 SNPs were prioritized for individual genotyping. All SNPs had same effect direction across all Euro populations. First step was to validate these in the pooled cohort.

FIG. 2. Pooled data analysis-EURO. 210 SNPs passed QC. 90% Call rate. HWE+0.05. MAF>5%. 88 SNPs had p-value <0.05.

FIG. 3. Pooled data analysis—FINLAND. 170 SNPs passed QC. 90% Call rate. HWE=0.05. MAF>5%. 156 SNPs had p-value <0.05.

FIG. 4. Compilation of human disease variants and other mutations UCSC known genes (June, 2005), based on UniProt, RefSeq, and GenBank mRNA.

FIG. 5. Immunohistochemistry results of mGluR7 (also referred to as GRM7) in mouse inner ear—a, b, c and f) GRM7 expression in spiral ganglion (SG) neurons (a), IHC (arrow) and OHCs (arrowheads) of the organ of Corti (a, f), hair cells of crista ampullaris (b) and sacculus (c) in adult mice—d, e, f) comparison of GRM7 expression in the IHC (arrow) and OHCs (arrowheads) of the organ of Corti at stage PD1 (d), PD21 (e) and in adult (f). Scale bar is 20 μm.

FIG. 6. Immunohistochemistry results of mGluR7 (also referred to as GRM7) in human temporal bone. (a) Anterior basal segment of horizontally cut temporal bone labeled with mGluR7 as the primary antibody and biotinolated IgG as the secondary antibody and fast red as the chromagen (MBIC). Hematoxylin (H) was used as a counter stain. Red labeling is visible in the interdental cells of the limbus, the hair and Hensen cells of the organ of Corti, and fibrocytes in the type II area or the spiral ligament. Moderate distortion is due to difficulty in mounting deceloidinized sections. (×100). (b) Higher power showing red labeling of interdental cells of the limbus. (MBIC & H×200). (c) Organ of Corti with labeled hair cells and Hensen's cells. (MBIC×400). (d) Spiral ganglion cells labeled with MBIC & H. (×400).

FIG. 7. Finemapping of the GRM7 locus on the European replication samples in the 400 kb region surrounding rs11928865 represents the finemapping results on both the training and the replication cohort combined. On top of the figure, the positions of the GRM7 exons within the 400 kb region are indicated with numbered vertical bars. The individual significances for each SNP are indicated with filled black circles on their respective locations within the GRM7 locus. The LD structure is illustrated at the bottom of the figures with the haplotype blocks numbered in brackets. The relative significances for each haplotype block are shown in the figure as well, with each horizontal numbered bar representing both the significance level and the location of each haplotype block.

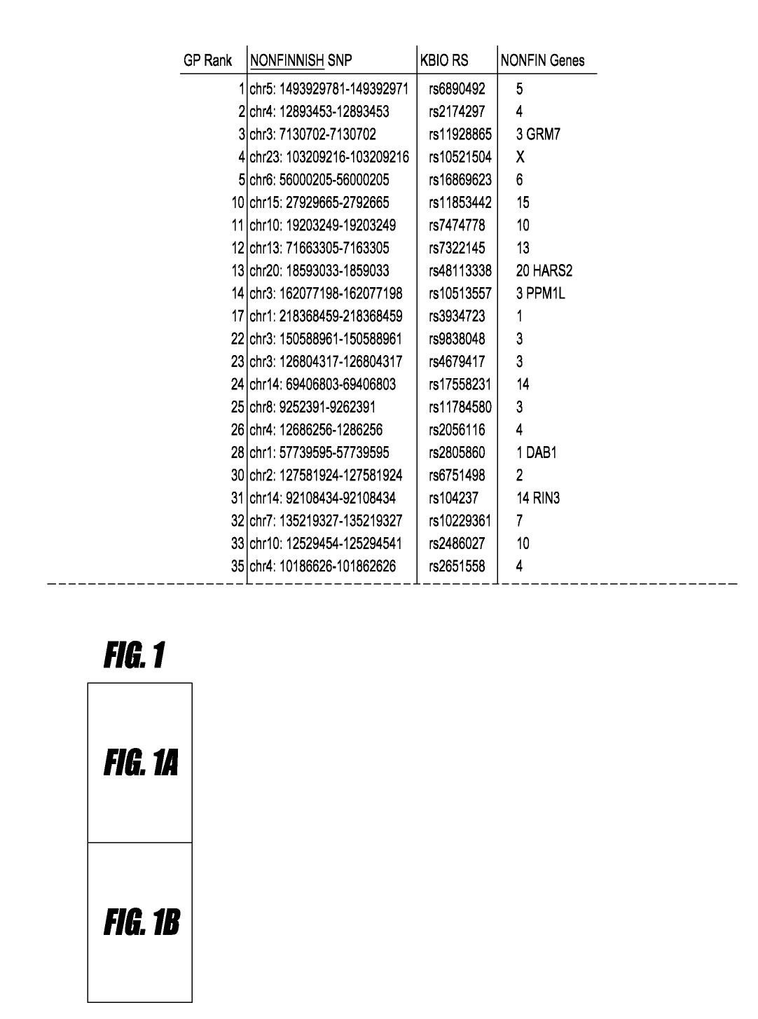

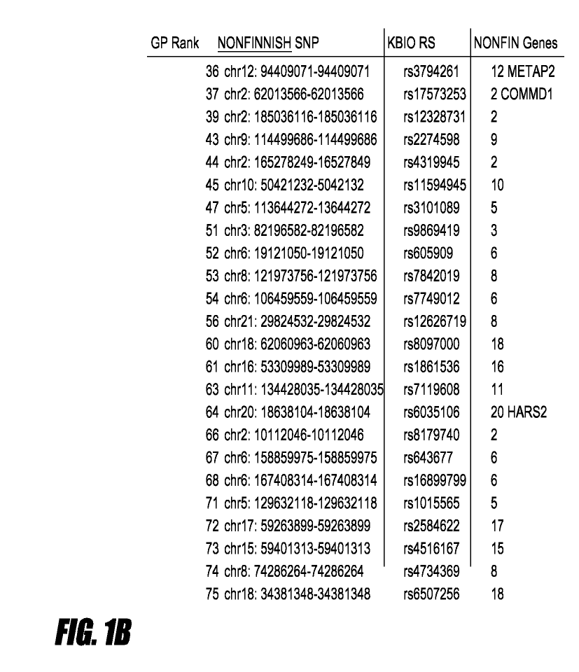

### DETAILED DESCRIPTION OF THE PREFERRED EMBODIMENT

In an attempt to better understand the genetics and biology underlying age-related hearing loss (presbycusis) and more particularly, to identify genes that may be related to presbycusis, and therefore, potentially available as therapeutic targets, a human genome scan was conducted using Affymetrix SNP arrays. mGluR7 (also referred to herein as GRM7) appeared in a very strong candidate region.

As with several other members of the mGluR family, mGluR7 is primarily localized on presynaptic terminals (Ohishi, H. et al. 1995 Neurosci. Lett. 202:85-88; Kinoshita, A. et al. 1998 J. Comp. Neurol. 393:332-352) where it is thought to regulate neurotransmitter release. Of the presynaptic mGluR subtypes, however, mGluR7 is the most widely distributed and is present at a broad range of synapses that are postulated to be critical for both normal CNS function and a range of human disorders. Furthermore, unlike some presynaptic mGluR subtypes, mGluR7 is localized directly in the presynaptic zone of the synaptic cleft of glutamatergic synapses (Kinoshita, A. et al. 1998 J. Comp. Neural. 393:332-35, Kosinski, C. M. et al. 1999 J. Comp. Neurol. 415:266-284), where it is thought to act as a traditional autoreceptor that is activated by the glutamate released from the presynaptic terminal during action potentials. mGluR7 has been thought to be a key player in shaping synaptic responses at glutamatergic synapses as well as in regulating key aspects of inhibitory GABAergic transmission (Kinoshita, A. et al. 1998 J. Comp. Neural. 393:332-35, Kosinski, C. M. et al. 1999 J. Comp. Neurol. 415:266-284). However, mGluR7 has been the most intractable of the mGluR subtypes in terms of discovery of selective ligands. Until the discovery of AMN082, there have been no selective agonists or antagonists of this receptor. Available agonists, such as L-AP4, activate mGluR7 only at concentrations 2-3 orders of magnitude higher than the concentrations required to activate its closest relatives, mGluRs 4, 6, and 8. Because of this, it has been impossible to study the physiological effects of activation of this receptor without confounding effects induced by activation of these related mGluR subtypes. Despite this lack of pharmacological tools, anatomical and cellular studies as well as experiments with mGluR7 KO mice have led to suggestions that selective ligands for this receptor have potential for treatment of a wide variety of neurological and psychiatric disorders, including depression, anxiety disorders, schizophrenia, epilepsy, Alzheimer's disease, and Parkinson's disease, among others.

Synaptic release of neurotransmitter in the nervous system is often influenced by presynaptic mGluRs which in turn respond to neurotransmitters released from the same nerve terminal or from terminals of other neurons. The mGluRs have diverse roles in synaptic plasticity such as long term potentiation (LTP) or long term depression, forms of synaptic plasticity believed to be involved in learning and memory in vertebrates. Presynaptic mGluR autoreceptors respond to glutamate and influence the probability of neurotransmitter release from a nerve terminal. In general, the activation of presynaptic mGluRs (e.g., class Ill mGluRs activated by L-AP4), has been found to reduce transmitter release from synapses in many brain regions.

Since their discovery, mGluRs have attracted attention as putative targets for many CNS indications, including anxiety, pain, neuroprotection, epilepsy, Parkinson's disease and cognitive disorders. Except for mGluR6, which is expressed exclusively on retina, all of the other mGluRs are expressed primarily on nerve terminals where they inhibit neurotransmitter release in the central and peripheral nervous systems (Shigemoto, R. et al. in: Handbook of Chemical Neuroanatomy Vol. 18, pp. 63-98, Ed. Ottersen Storm-Mathisen, Amsterdam, Elsevier). It has been proposed that mGluRs possess a large bi-lobed extracellular N-terminus, where glutamate binds within a cleft between the two lobes and stabilizes a closed conformation (Yang, Z.-Q. 2005 Curr. Topics Med. Chem. 5:913-918). Studies also suggest that C-terminus of mGluRs also play an important role in affecting receptor-ligand binding.

The general structure of the metabotropic receptors consists of a glutamate binding site, a cystein-rich region, a seven transmembrane domain and an intracellular C-terminal region. The latter domain has extensively been investigated in isoform GRM7_v1 and appears to be divided into three functional regions. The most proximal encompassing amino acid (aa) residues 856-878 plays a role in the signaling complex, the central part (aa 883-915) is involved in the axonal targeting while the immediate C-terminus (aa 912-915) is involved in the synaptic clustering of the receptor via 95 kDa postsynaptic density protein (PSD-95)/discs-large (Dlg)/zona occludens-1 (ZO-1) (PDZ) domain (Dev, K. K. et al. 2001 Trends Pharmacol. Sci. 22:355-361).

As discussed above, one member of the mGluR family, mGluR7, remains poorly characterized. It is the most conserved member of the mGluR family, with only 6-8 amino acid differences observed between rat and human proteins. mGluR7 is widely distributed throughout the nervous system and is localized presynaptically close to neurotransmitter release sites. In the hippocampus, a high density of the mGluR7a isoform is found, in particular, in presynaptic terminals of excitatory cells that synapse on mGluR1α expressing GABAergic interneurons that also express somatostatin. Therefore, the input of this particular class of interneuron seems to be endowed by a particularly strong mGluR7-mediated autoregulation. Why this is so remains speculative for the moment and it has been suggested that this peculiar mGluR7-mediated autoregulation might relate to the role of the mGluR1α₁-positive interneurons in the hippocampal network (Shigemoto et al. 1996 Nature 318:523-525). mGluR1α⁺ cells receive glutamatergic input from axon collaterals of principal cells. The GABAergic terminals of mGluR1α⁺ cells make synapses on principal cell distal dendrites, which also receive a direct excitatory input from the entorhinal cortex.

mGluR7 (also referred to as GRM7) was originally cloned from a rat forebrain complementary DNA (cDNA) library (Okamoto, N. et al. 1994 J. Biol. Chem. 269:1231-1236). Subsequently, the human homologue was identified (Makoff, A. et al. 1996 Mol. Brain. Res. 40:165-170, GenBank Accession Number CAA64245) and later two isoforms, GRM7_vI and GRM7_v2 (formerly mGlu7a and mGlu7b) were reported (Flor, P. J. et al. 1997 Neuropharinacol. 36:153-159, GenBank Accession Numbers NP_{—}000835 and NP_{—}870989). The two transcript variants differ by an out-of-frame insertion of 92 by at the 3′ end of the coding region resulting in two putative proteins of 915 and 922 aa with distinct C-termini. While the GRM7_v1 isoform is expressed throughout the CNS (Kinoshita, A. et al. 1998 J. Comp. Neural. 393:332-352), GRM7_v2 localization in the brain appears to be more restricted and is preferentially found in distinct regions such as hippocampus, ventral pallidum, and globus pallidus (Kinoshita, A. et al. 1998 J. Comp. Neurol. 393:332-352). Both isoforms are localized to the active zones of presynaptic axon terminals close to the glutamate release site (Kinoshita. A. et al. 1998 J. Comp. Neurol. 393:332-352; Saugstad, J. A. et al. 1994 Mol. Pharmacal. 45:367-372). Recently, Schultz, H. L. et al. (2002 Neurosci. Lett. 326:37-40) identified three novel isoforms of the mGluR7, termed GRM7_v3, GRM7_v4, and GRM7_v5 (GenBank Accession Numbers: AF458052, AF4458053 and AF458054, respectively).

Recently, rapid progress has been made on development of new agonists and antagonists of mGluRs (Schoepp, D. D. et al. 1999 Neuropharmacol. 38:1431-1476). However, the therapeutic utilities of most of these ligands are hampered by their lack of selectivity for each subtype and poor CNS penetration. Most known mGluR agonists show no or very little activities at mGluR7. The active of isomer of (R,S)-phosphonophenylglycine (PPG), (+)—PPG, had been until recently the most potent agonist at mGluR7, with an EC₅₀ value of 48 μM on human mGluR7b (Yang, Z.-Q. 2005 Curr. Topics Med. Chem. 5:913-918). However, (+)-PPG was found more active at mGluR4a (EC₅₀=3.2 μM).

The compound described by Mitsukawa et al. (2005 PNAS USA 102:18712-18717), called AMN082, was identified by using a random high-throughput screen (HTS) of a library of small drug-like molecules in search of novel compounds that activate mGluR7. From this screen and subsequent experiments, AMN082 emerged as a highly selective and potent agonist of mGluR7. In addition to providing the first selective ligand for this important receptor subtype. AMN082 acts by a unique mechanism of action, fully activating mGluR7 through an allosteric site far removed from the glutamate-binding pocket. The compound is structurally unrelated to any known mGluR ligand and provides an excellent example of the power of HTS in identifying novel compounds that are unrelated to known chemical scaffolds. Remarkably, this primary HTS hit was found to be orally active and to penetrate the blood-brain barrier. Indeed, AMN082 induced a robust increase in stress hormone levels that was absent in mGluR7 knockout animals, providing powerful support to a growing set of findings that suggest that antagonists of this receptor may be useful in conditions involving chronic stress such as depression and anxiety disorders. However, the implications of these studies go far beyond the role of mGluR7 in stress responses.

The carboxylic derivative of L-CCG-I (2S,1'S,2'S)-2-(dicarboxycyclopropyl)glycine [DCG-IV], is a group II selective agonist. It is also an antagonist of Group III mGluRs. The 10₅₀ values on inhibition of glutamate stimulated inositol phosphate accumulation are reported as 39.6, 40.1 and 32 μM at mGluR6, 7 and 8, respectively. Similarly, LY341495, a xanthen-substituted L-CCG-I, a selective antagonist of Group II mGluRs, also displays potent antagonist activities at mGluR5, 7 and 8. The Kd value measured for binding of [3H]-LY341495 for mGluR7 is 0.0727 mM.

Recently, Suzuki et al. (2007 JPET 323:147-156) reported the discovery and pharmacological characterization of novel isoxazolopyridone derivatives that are metabotropic glutamate receptor (mGluR) 7 antagonists. 5-Methyl-3,6-diphenylisoxazolo[4,5-e]pyridin-4(5H)-one (MDIP) was identified by random screening, and 6-(4-methoxyphenyl)-5-methyl-3-pyridin-4-ylisoxazolo[4,5-c]pyridin-4(5H)-one (MMPIP) was produced by chemical modification of MDIP. MDIP and MMPIP inhibited L-(+)-2-amino-4-phosphonobutyric acid (L-AP4)-induced intracellular Ca²⁺ mobilization in Chinese hamster ovary (CHO) cells coexpressing rat mGluR7 with G15 (IC₅₀=20 and 26 nM). The maximal response in agonist concentration-response curves was reduced in the presence of MMPIP, and its antagonism was reversible. MMPIP did not displace [3H](2S)-2-amino-2-[(1S,2S)-2-carboxycycloprop-1-yl]-3-(xanth-9-yl) propanoic acid (LY341495) bound to mGluR7. These results suggested that these isoxazolopyridone derivatives are allosteric antagonists. In CHO cells expressing rat mGluR7, MDIP and MMPIP inhibited L-AP4-induced inhibition of forskolin-stimulated cAMP accumulation (IC₅₀=99 and 220 nM). In CHO cells coexpressing human mGluR7 with G15, MDIP and MMPIP also inhibited the L-AP4-induced cAMP response. The maximal degree of inhibition by MMPIP was higher than that by MDIP in a cAMP assay. MMPIP was able to antagonize an allosteric agonist, the N,N′-dibenzhydryl-ethane-1,2-diamine dihydrochloride (AMN082)-induced inhibition of cAMP accumulation. In the absence of these agonists, MMPIP caused a further increase in forskolin-stimulated cAMP levels in CHO cells expressing mGluR7, whereas a competitive antagonist, LY341495, did not. This result indicates that MMPIP has an inverse agonistic activity. The intrinsic activity of MMPIP was pertussis toxin-sensitive and mGluR7-dependent. MMPIP at concentrations of at least 1 μM had no significant effect on mGluR1, mGluR2, mGluR3, mGluR4, mGluR5, and mGluR8. MMPIP is the first allosteric mGluR7-selective antagonist that could potentially be useful as a pharmacological tool for elucidating the roles of mGluR7 on central nervous system functions. Additional mGlur7 antagonists may be selected from the compounds described in U.S. Pat. No. 7,053,219.

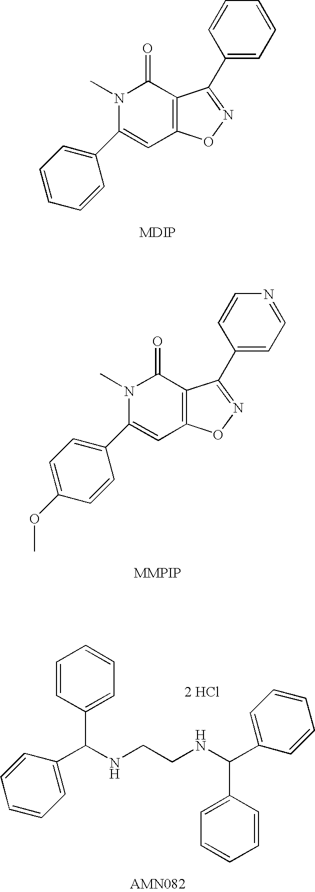

Although the precise physiological functions of the GRM7 subtypes are still unclear, targeted disruption of the orthologous murine Grm7 gene locus has been shown to cause a deficit in fear response and an impairment of taste aversion. This suggests a critical role of Grm7 in amygdala function which is essential in relating these behavioral traits (Masugi, M. et al. 1999 J. Neurosci. 19:955-963). Upon drug induction mice lacking Grm7 are susceptible to epileptic seizures indicating that Grm7 may be particularly important in the regulation of neuronal excitability (Sansig, G. et al. 2001 J. Neurosci. 21:8734-8745).

In preferred embodiments, the invention relates to use of pharmaceutical compounds that act specifically as mGluR7 receptor modulators to treat or prevent presbycusis.

In one embodiment, the invention relates to a method for treating presbycusis in a human. The method comprises administering to a human a therapeutically effective amount of a pharmaceutical composition comprising an mGluR7 receptor modulator. The mGluR7 receptor modulator is administered in an amount and for a period of time, effective to reduce or alleviate the symptoms of presbycusis in a human in need of such treatment.

In another embodiment, the invention relates to a method for preventing presbycusis in a human. This method comprises administering to a human a therapeutically effective amount of a pharmaceutical composition comprising an mGluR7 receptor modulator. In this method the mGluR7 receptor modulator is administered in an amount and for a period of time, effective to prevent presbycusis in an individual in need of such treatment.

### Administration and Formulation

Delivery of the compound to patients can be accomplished orally, intravenously, subcutaneously, intraperitoneally, intramuscularly, rectally or topically, whereas topical administration to the inner ear is generally preferred, as therapeutically effective doses with systemic administration may induce undesired side-effects. One of skill in the art will recognize that administration of an mGluR7 receptor modulator in the present invention may be accomplished in a variety of other ways. The only requirement for administration in the present invention is that a therapeutically effective amount of a pharmaceutical composition comprising an mGluR7 receptor modulator be able to reach the site of the mGluR7 receptor mediated aberrant activity in the afflicted individual.

Administration of the compound to the inner ear may be accomplished by various delivery techniques. These include the use of devices or drug carriers to transport and/or deliver the compound in a targeted fashion to the membranes of the round or oval window, where it diffuses into the inner ear or is actively infused. Examples are otowicks (see e.g., U.S. Pat. No. 6,120,484 to Silverstein), round window catheters (see e.g., U.S. Pat. Nos. 5,421,818; 5,474,529; 5,476,446; 6,045,528; all to Arenberg, or 6,377,849 and its division 2002/0082554 to Lenarz), or various types of gels, foams, fibrins or other drug carriers, which are placed in the round window niche or on the oval window, and loaded with the compound for sustained release (see e.g., WO 97/38698 by Manning; Silverstein et al. 1999 Otolaryagology-Head and Neck Surgery 120:649-655; Balough et al. 1998 Otolaryngology-Head and Neck Surgery 119:427-431). They further include the use of devices which are inserted into the cochlear duct or any other part of the cochlea (see e.g., U.S. Pat. No. 6,309,410 to Kuzma). The compound may also be administered to the inner ear by transtympanic injection, where the middle ear or part of it is filled by a solution or other carriers of the compound (see e.g., Hoffer et al. 2003 Otolaryagologic Clinics of North America 36:353-358). The preferred method of administration to the inner ear is by diffusion across the round window membrane, which is relatively easily accessible from the middle ear space, and allows the inner ear to remain intact, thus avoiding any potential problems from leaking intracochlear fluids.

In preferred embodiments, the compounds can be provided in any of a variety of formulations compatible with delivery across a middle-inner ear membrane, provided that such formulation is stable (i.e., not subject to degradation to an unacceptable amount at body temperature). The compound can be provided in any form suitable for delivery and diffusion of agent across the middle-inner ear membrane structure, e.g., solid, semi-solid, gel, liquid, suspension, emulsion, osmotic dosage formulation, diffusion dosage formulation, erodible formulation, etc. In one embodiment, the formulation is suitable for delivery using an implantable pump in connection with a catheter inserted near the round window niche of the inner ear, e.g., an osmotic pump.

Pharmaceutical grade organic or inorganic carriers, excipients and/or diluents can be included in the formulations. The formulations can optionally comprise a buffer such as sodium phosphate at physiological pH value, physiological saline or both (i.e., phosphate buffered saline). Suitable excipients can comprise dextrose, glycerol, alcohol (e.g., ethanol), and the like, and combinations of one or more thereof with vegetable oils, propylene glycol, polyethylene glycol, benzyl alcohol, benzyl benzoate, dimethyl sulfoxide (DMSO), organics, and the like to provide a suitable composition. In addition, if desired, the composition can comprise hydrophobic or aqueous surfactants, dispersing agents, wetting or emulsifying agents, isotonic agents, pH buffering agents, dissolution promoting agents, stabilizers, antiseptic agents and other typical auxiliary additives employed in the formulation of pharmaceutical preparations. The compound can be provided in the formulation as a solution, a suspension, and/or as a precipitate.

A compound contained within the pharmaceutical composition of this invention may be provided in the form of a pharmaceutically acceptable salt. Examples of such a salt include, but are not limited to, those formed with organic acids (e.g., acetic, lactic, citric, malic, formaric, tartaric, stearic, ascorbic, succinic, benzoic, methanesulfonic, tolu-enesulfonic, or pamoic acid), inorganic acids (e.g. hydrochloridic, nitric, diphosphoric, sulphuric, or phosphoric acid), and polymeric acids (e.g., tannic acid, carboxymethyl cellulose, polylactic, polyglycolic, or co-polymers of polylactic-glycolic acids).

Pharmaceutical compositions for any route of administration of this invention contain a therapeutically effective amount of active ingredient, and, as may be necessary, inorganic or organic, solid or liquid pharmaceutically acceptable carriers. Pharmaceutical compositions suited for topical administration to the inner ear include aqueous solutions or suspensions, which, e.g., in the case of lyophilized formulations that contain the active ingredient alone or together with a carrier, may be prepared prior to use. They further include gels, which may be biodegradable or non-biodegradable, aqueous or non-aqueous, or microsphere based. Examples of such a gel include, but are not limited to, poloxamers, hyaluronates, xyloglucans, chitosans, polyesters, poly(lactides), poly(glycolide) or their co-polymers PLGA, sucrose acetate isobutyrate, and glycerol monooleate. Pharmaceutical compositions suited for enteral or parenteral administration include tablets or gelatine capsules or aqueous solutions or suspensions as described above.

The pharmaceutical compositions may be sterilized and/or may contain adjuvants, e.g., preservatives, stabilizers, wetting agents and/or emulsifiers, salts for regulating the osmotic pressure and/or buffers. The pharmaceutical compositions of the invention may, if desired, contain further pharmacologically active substances. They may be prepared by any of the methods well known in the art of pharmacy, e.g., by conventional mixing, granulating, confectioning, dissolving or lyophilizing methods, and contain from about 0.01 to 100%, preferably from about 0.1 to 50% (lyophilisates up to 100%), of active ingredient.

In a preferred embodiment the pharmaceutical composition is formulated for topical application. Suitable vehicles for otic administration are organic or inorganic substances, which are pharmaceutically acceptable and which do not react with the active compounds, for example saline, alcohols, vegetable oils, benzyl alcohols, alkylene glycols, polyethylene glycols, glycerol triacetate, gelatin, carbohydrates such as lactose or starch, magnesium, stearate, talc and petrolatum. The indicated preparations can be sterilized and/or contain ancillary substances such as lubricants, preservatives, such as thimersal (e.g., at 50%), stabilizers and/or wetting agents, emulsifiers, salts to influence the osmotic pressure, buffer substances, colorants, and/or aromatizing substances.

The pharmaceutical compositions may also contain one or more other active ingredients. Otic compositions in accordance with aspects of the present invention can comprise various ingredients, including other biologically active agents, such as antibiotics, e.g., fluoroquinolones, anti-inflammatory agents, e.g., steroids, cortisone, analgesics, antipyrine, benzocaine, procaine, etc. In one embodiment, the pharmaceutical composition may contain a combination of an mGluR7 modulator and an iGluR modulator, such as the NMDA receptor antagonists disclosed in US 2007/0015272, including but not limited to D-2-amino-5-phosphonopentanoate (D-AP5), Dizocilpine (MK 801), 7-chlorokynurenate (7-CK) and Gacyclidine (GK-11).

Compositions in accordance with preferred embodiments of the present invention for topical administration may comprise other ingredients which are pharmaceutically acceptable. In preferred embodiments of the present invention, a topical excipient is selected that does not enhance delivery of the agent to the systemic circulation or to the central nervous system when administered to the ear. For example, in general, it is preferred that the topical excipient not have substantial occlusive properties, which enhance percutaneous transmission through the mucosa into the systemic circulation. Such occlusive vehicles include hydrocarbon bases, anhydrous absorption bases such as hydrophilic petrolatum and anhydrous lanolin (e.g., Aquaphor), and water-in-oil emulsion bases such as lanolin and cold cream. More preferred are vehicles which are substantially non-occlusive, and generally include those which are water soluble, such as oil-in-water emulsion bases (creams or hydrophilic ointments) and water soluble bases such as polyethylene glycol-based vehicles and aqueous solutions gelled with various agents such as methylcellulose, hydroxyethyl cellulose, and hydroxypropyl methylcellulose (e.g., KY Gel).

Suitable topical excipients and vehicles can be routinely selected for a particular use by those skilled in the art, and especially with reference to one of many standard texts in the art, such as Remington's Pharmaceutical Sciences, Vol. 18, Mack Publishing Co., Easton, Pa. (1990), in particular Chapter 87. For instance, biologically active agents in accordance with aspects of the present invention can be combined with enhancing agents which enhance the penetration of an agent.

The pharmaceutical compositions can be administered prior to development of presbycusis, or after presbycusis has been diagnosed. The amount to be administered may vary, depending upon the method of administration, duration of therapy, the condition of the subject to be treated, the severity of presbycusis and the efficacy of the particular compound used, age, body weight, general state of health, sex, diet, time and route of administration, rate of excretion and drug combination ultimately will be decided by the attending physician. The duration of therapy may range between about one hour and several days, weeks or months, and may extend up to chronic treatment. The therapeutically effective amount of the pharmaceutical compositions to be delivered may range between about 0.1 nanogram/hour to about 100 micrograms/hour. The pharmaceutical compositions are preferably administered analogously to other otically administered compounds. By the term “dosage” for topical administration, it is meant the amount of agent administered in a single treatment, e.g., about 0.05-1 μg of the mGluR7 receptor modulator administered to the ear in two drops.

A therapeutically effective dose is defined as an amount effective to suppress or reduce presbycusis in the afflicted individual. As stated above, a therapeutically effective dose may vary, depending on the choice of specific mGluR7 receptor modulator for treatment and on the method of its administration. For example, a higher dose of an intravenously administered mGluR7 receptor modulator would be required than that of the same pharmaceutical composition administered locally to the round window membrane or oval window of the ear. Additionally, a lower dose of an mGluR7 receptor modulator would be required wherein the mGluR7 receptor modulator of the present invention binds the mGluR7 receptor with a higher binding affinity than an mGluR7 receptor modulator that binds with a lower affinity. As a result, mGluR7 receptor modulators with higher binding affinities for the mGluR7 receptor are preferred.

The duration of therapy may also vary, depending on the specific form of presbycusis for which treatment is desired—acute, subacute, or chronic. As a guide, shorter durations of therapy are preferred and are sufficient when the presbycusis does not recur once therapy has ceased. Longer durations of therapy may be employed for an individual in which presbycusis persists following short therapy.

The inventive methods disclosed herein for treating or preventing presbycusis may allow analogously the use of a therapeutically effective amount of a pharmaceutical composition comprising an mGluR7 receptor modulator, advantageously effective to modulate mGluR7 receptor mediated aberrant activity in a human, for the manufacture of a medicament for the treatment or prevention of presbycusis.

### Example 1

Whole Genome Association Study (“WGAS”) was powered to detect common variants. The common disease common variant (CDCV) hypothesis rests on the fact that the single nucleotide polymorphisms (SNPs) (markers) assayed arose through evolution and may have been protective in previous times. For WGAS, we utilized “unrelated” individuals=unknown distant relationships. Because of the large time span under study we cannot collect samples from each generation. We relied on statistical correlations between phenotype and genotype. Rather than compare the entire genetic code of individuals with and without disease, we relied on surrogate markers scattered across the human genome. The most commonly used markers are termed single nucleotide polymorphisms (SNPs).

Presbycusis, as a disease, likely conforms to the CDCV hypothesis because it is a common disease that manifests later in life. Therefore any SNP marker that might be associated with disease (and therefore one that is assayed during our experiments) likely had no negative selection pressure during human history. Lastly, presbycusis is known to have a genetic aspect. Therefore, presbycusis was an excellent candidate for the WGAS.

### WGAS Design

Clinical phenotyping and collection of case and control individuals matched for ethnicity (see TABLE 1).

Quality control of starting material (DNA).

Whole genome association study at ˜500,000 SNP markers.

Comprehensive analysis of this large amount of data for association.

Investigation of top significant SNPs in a unique clinical cohort(s).

Biological experiments to support the validated genetic findings.

Completely independent validation of the findings in a new cohort.

### Clinical Criteria

We calculated how many standard deviations the recorded hearing threshold diverged from the median threshold value (Z-score). Specific for age (between 55-65), gender, and frequency. We used the average of the Z-scores from 2, 4, and 8 kHz. Pure-tone thresholds with air and bone conduction were registered at 0.125, 0.25, 0.5, 1, 2, 4, and 8 kHz.

### Exclusions

A conductive component (a mean air-bone gap at 0.5, 1, and 2 kHz exceeding 10 dB).

A dip at 4 kHz (if the 4 kHz threshold exceeded the 8 kHz threshold by 20 dB or more).

A disease that may influence hearing: chronic otitis media, auto-immune disease, chemotherapy, rheumatoid arthritis.

TABLE 1

| | TOTAL | | CON- | |
|---|---|---|---|---|
| LOCATION | SAMPLES | CASE | TROL | QUARTILE |
| Antwerp, Belgium | 984 | 192 | 192 | 19.9 |
| Oulu, Finland | 505 | 96 | 96 | 19.4 |
| Copenhagen, Denmark | 404 | 96 | 96 | 24.2 |
| Tubingen, Germany | 395 | 96 | 96 | 24.8 |
| Padova, Italy | 355 | 96 | 96 | 29.4 |
| Gent, Belgium | 342 | 96 | 96 | 28.5 |
| Nijmegen, Nederlands | 276 | 90 | 90 | 33.0 |
| Tampere, Finland | 258 | 84 | 84 | 33.0 |
| TOTALS | | 846 | 846 | |

### Quality Check

Samples were checked by gel electrophoresis to identify any degraded DNA. None were found.

### Quantitation

PicoGreen assay: Each sample was quantitated in triplicate (1,859×3=5,577 samples quantitated). Concentration of samples was determined from an 8-point standard curve with human genomic DNA: 200, 100, 50, 25, 12.5, 6.25, 3.125, and 0 ng/μl. Sample concentrations were calculated using the median value from the 3 replicates. Samples were flagged for re-PicoGreening if any of the 3 values were more than double the others. Upon re-PicoGreening most samples were successfully quantitated. Only 6 samples total were excluded out of the entire 1,859 samples.

### Experimental Design

EURO Cohort→pooled genotyping→pooling validation cross-genotyping.

FINLAND cohort→pooled genotyping→pooling validation cross-genotyping.

200 ng of each sample was pipetted into 3 (triplicate) pools of cases and controls of each population. 846 Cases+846 Controls=1,692 samples in total were used in pools. 8 populations×2 cohorts×pooled in triplicate (to control for pipetting error)=48 pools. To control for chipping error, 48 pools created in triplicate→144 pools. 144 pools were digested with Nsp I and Sty I→288 samples for chipping.

Each SNP was scored within each sub-population using 4 different algorithms (GenePool). SNPs were examined using single marker examinations and sliding window analyses. SNPs were ranked based on overall median rank of the algorithms. Lastly, allele frequencies were calculated and SNPs showing opposite allele effect were removed.

The “Euro” population was examined separately from the Finnish population—largest ever at approximately 70 Million SNP genotypes.

The pooled WGAS approach was able to successfully prioritize regions across the genome that were significantly associated with presbycusis.

### Cross-Genotyping Results

No SNP survived with significance when it was genotyped in the cross-genotyping round. What does this mean? We know that Finland and the Europe populations have differing genetic backgrounds—this is why the initial analysis was done separately. Therefore we don't necessarily expect the exact same marker to be associated with disease. However, we might predict that if there was a major gene contributing to presbycusis we should detect it in both populations.

### EURO Replication

We tested a select list of SNPs in a novel population recruited from Antwerp and Tubingen. To be selected, a SNP must: (1) have a significance of less than 0.003 in the original Euro pooled cohort, (2) have a significance of less than 0.05 in the Antwerp cohort alone, and (3) reside within a gene. This resulted in a selection of 23 SNPs from the original list of 210. The novel replication cohort consisted of individuals that were phenotyped exactly as the original pooling cohort. Again, they were the extreme quartiles based upon z-scores. Cohort consisted of 61 cases and 66 controls. SNPs were included for analysis using same criteria as before, >90% Call Rate, HWE=0.05, MAF=5%. 19 SNPs survived these cut-offs.

rs11928865 SNP survived at least 10,000× permutes of the data [p=0.0038]; TABLE 2. It ranks #3 in the Euro population pooled analysis. It is located within the GRM7 locus—note that there are two SNPs located within GRM7 that are in the top 200 in the Finland pooled analysis.

TABLE 2

| SNP ID | ALLELE | FREQ | P-value | POPULATION |
|---|---|---|---|---|
| rs11928865 | T | 77.0% CA; | 0.0003 | EURO POOL |
| | | 71.1% CO | | |
| rs11928865 | T | 78.7% CA; | 0.0003 | EURO REPL |
| | | 59.1 CO | | |
| rs11928865 | — | T: 72.5%; | — | CEPH |
| | | A: 27.5% | | |
| rs779701 | — | — | 0.0058 | FIN POOL |
| rs779706 | — | — | 0.0067 | FIN POOL |

rs11928865; chr3: 7,130,702
rs779701; chr3: 7,493,772
rs779706; chr3: 7,499,042

In summary, a total of 846 cases and 846 controls out of 3434 individuals collected by 8 centers from various European regions, including Finland, were analyzed in this study (TABLE 1). Case and control pools were created in triplicate for each subpopulation (n=8) and each pool was genotyped on three replicate Affymetrix Human Mapping 500K arrays, containing 506627 SNPs.

SNPs from the pooled whole genome association study were ranked. Each subpopulation was evaluated separately, followed by an analysis of all non-Finnish European samples combined. The samples collected from Finland (Oulu and Tampere) were considered to be genetically distinct from the remaining European samples and were analyzed as a whole separately. For the most highly ranked SNPs we verified whether the associated risk allele was the same throughout all subpopulations. If the associated allele was not the same in all subpopulations, that SNP was excluded from further study. The top 252 SNPs identified in the European population study and the top 177 SNPs identified in the Finland-based study, ranked by the GenePool software and fulfilling all criteria, were taken forward for validation by individual genotyping. The resulting individual-based x-square p-values are listed in TABLE 2. The individual genotyping experiment comprised a clear validation of the pooling approach. DTD1 (D-tyrosyl-tRNA deacylase 1) and PDE9A (phosphodiesterase 9A) were the top-ranked genes for the general European population and the Finnish population, respectively. Two genes were significantly associated in both populations: GRM7 (glutamate receptor, metabotropic, 7) and CDH13 (cadherin 13).

Subsequently, a select sub-set of 23 SNPs was examined in a European replication cohort consisting of 142 newly recruited individuals. SNPs were only included if they passed strict criteria, including (1) being within a gene with five exceptions, (2) passing HWE measurements at a cut-off of 0.05, (3) exhibiting an individual call rate >97%, and (4) having a minor allele frequency of a least 5%. In order to correct for multiple testing, we applied Bonferroni correction. Due to linkage disequilibrium (LD), these 23 SNPs represented only 20 true tests, yielding a Bonferroni corrected significance p-value of 0.0025. One SNP of GRM7 (rs11928865) yielded a p-value of 0.0003 in the replication cohort, and thus survived the Bonferroni correction. Permutation testing gave a p-value of 0.0044.

In both training and replication cohorts the T-allele of rs11928865 was associated with disease. The frequencies were 76.9% in cases and 71.4% in controls for the training cohort, and 79.3% in cases and 57.8% in controls for the replication cohort. The odds ratio for this SNP was 2.56 [95% confidence interval of 1.23-5.30] in the replication cohort. As no Finnish replication cohort could be obtained, the SNPs within GRM7, significant in the Finnish population, rs779706 and rs779701, could not be replicated.

### GRM7 Fine Mapping

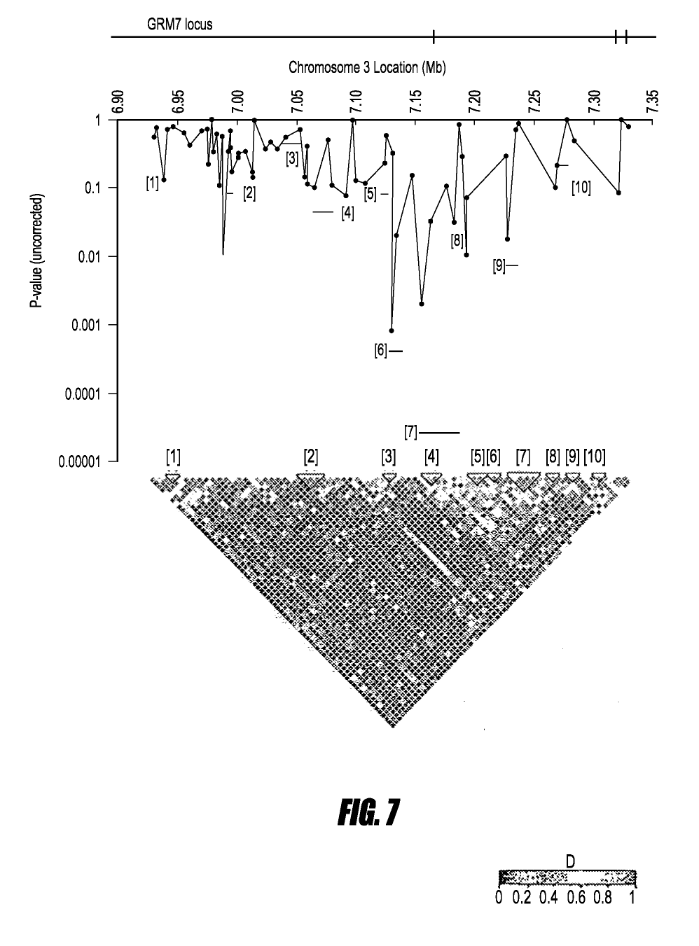

GRM7 measures approximately 880 kb. Fine mapping was restricted to a region of 400 kb surrounding rs11928865. Based on hapmap data, 80 GRM7 tag SNPs were selected and genotyped on all European samples (training and replication cohort). FIG. 7 illustrates the fine mapping of the GRM7 locus in the European replication samples. Rs11928865 remained the most significantly associated individual SNP, while haplotype block 7 (consisting of SNPs rs6804466, rs3828472, rs12497688, rs12497688, rs9819783, rs11920109) and 6 (consisting of SNPs rs11928865 and rs9877154) were the most significantly associated haplotype blocks (TABLE 2). One million permutations of the data resulted in a p-value of 0.0013 and 0.0266 for haplotype blocks 7 and 6, respectively. Because haplotype blocks 6 and 7 were in close proximity and because of the fairly high level of LD between them, we forced both blocks into a single block. This joined 6-7 haplotype has a p-value of 0.0004 which was the most significant of all permuted p-values. We have genotyped a large number of closely spaced SNPs across the GRM7 gene locus to whittle the association signal down to a narrow region, and this indicated that the causative allele is positioned within a 150 kb region surrounding GRM7 exon 2.

We completed the largest pooling-based whole genome association study to date to uncover the genetic risk factors for presbycusis. We have identified a SNP residing in GRM7 that was significant in the original pooled EURO cohort and significant in the validation EURO cohort. Additionally, two SNPs within the same gene were significant in the Finland based cohort. The biology surrounding mGluR7 provides support for the mGluR7 having an important role in presbycusis.

### Example 2

### Materials and Methods

### Cloning of GRM7 Fragment and Probe Synthesis for In Situ Hybridisation

Mouse brain RNA was isolated using Trizol (Invitrogen, Carlsbad, Calif., USA) and reverse-transcribed with the Superscript™ III First-strand synthesis system for RT-PCR (Invitrogen, Carlsbad, Calif., USA). A 839 bp-fragment of mouse GRM7 (nucleotides 901 to 1739, based on (Kosinski C. M. et al. 1999 J. Comp. Neurol. 415:266-84) was amplified using the iProof High-fidelity DNA polymerase kit (BioRad, Hercules, Calif., USA) according to the manufacturer's instructions, in the presence of 200 μM dNTP mix (BD Biosciences Clontech, Palo Alto, Calif., USA) and 0.5 μM of each primer (5′-AGAGCTGACCAAGTAGGAC-3′, SEQ ID NO: 1 and 5′-GGGATGTTCTGACAGCCAG-SEQ ID NO: 2)(Invitrogen, Carlsbad, Calif., USA). PCR product was gel-purified using the QIAquick Gel Extraction kit (QIAGEN GmbH, Hilden, Germany) and 3′A-overhangs were generated by adding 1×PCR-buffer, 200 μM dNTP mix (BD Biosciences Clontech, Palo Alto, Calif., USA) and OA U/μl of Silverstar™ Taq polymerase (Eurogentec, Seraing, Belgium), followed by an incubation at 72° C. for 10 min. The fragment was subsequently cloned with the TOPO TA Cloning kit for sequencing (Invitrogen, Carlsbad, Calif., USA), following the manufacturer's protocol. Inserts were verified by sequencing. Plasmids were linearized with either NotI or SpeI (Fermentas GmbH, St. Leon-Rot, Germany) for 3 hours at 37° C. and digests were purified using the Rapid PCR purification system (Marligen Bioscience Inc, Ijamsville, Md., USA). Subsequently, digoxigenin (DIG)-labeled antisense and sense riboprobes were generated with T3 and T7 polymerase using Riboprobe™ in vitro Transcription Systems (Promega, Madison, Wis., USA) and DIG-11-UTP (Roche Diagnostics, Brussels, Belgium) as prescribed by the manufacturer. Riboprobes were then hydrolized to a fragment length of approximately 150 bp, for better diffusion into the tissue. After ethanol precipitation, pellets were dissolved in diethylpyrocarbonaat (DEPC)-treated water. Probe concentrations were determined using the DIG Nonradioactive Nucleic Acid Labeling and Detection System (Roche Diagnostics, Brussels, Belgium) according to the instructions of the manufacturer.

### Tissue Preparation

Mouse inner ear tissue from postnatal day (PD) 1, PD21 and adult mice (age ranging from PD42 to PD70) was prepared as described previously (Cryns K. et al. 2003 Histochem Cell Biol. 119:247-56). Briefly, mice were transcardially perfused, inner ears were removed and postfixed in 4% phosphate buffered paraformaldehyde (PF/PB). After fixation, P21 and adult inner ears were decalcified in phosphate buffered saline (PBS) containing 5% ethylenediaminetetraacetic acid (EDTA). Tissue was paraffin embedded and 5 μm-thick sections were mounted on uncoated glasses and used for immunohistochemistry.

### In Situ Hybridization

In situ hybridization on tissue sections was performed with modifications as described (Groves and Bronner-Fraser, 2000 Development 127:3489-99). Briefly, antisense digoxigenin-labeled RNA probes were synthesized according to the Riboprobe™ System protocol (Promega Co., Madison, Wis.). Sections were treated with 2 μg/ml proteinase K in PK buffer (pH 7.5) at room temperature for 10 min. The tissue sections were then delipidated and dehydrated before hybridization. Hybridization with digoxigenin-labeled riboprobes was performed at 60° C. overnight in a hybridization buffer solution. After hybridization, sections were incubated in 20% heat-inactivated sheep serum in PBT to block non-specific binding sites. To visualize the hybrids, the sections were incubated with an anti-digoxigenin antibody (conjugated with alkaline phosphatase). The slides were developed in the dark for 24 hours at room temperature after the addition of a chromogenic substrate, BCIP using NBT as a catalyst.

Three to five embryos of each genotype were analyzed for every probe. Immunohistochemistry

Paraffin sections of mouse inner ear were deparaffinized with xylol and rehydrated in series of ethanol. Sections were washed in PBS and endogenous avidin and biotin were blocked by endogenous avidin/biotin blocking kit (Zymed Laboratories, San Francisco, Calif., USA), as described by the manufacturer, followed by extensive rinsing. To avoid aspecific binding from the primary antibody, sections were blocked with freshly prepared solution of 50 mM glycin/PBS for 15 min and rinsed once more. Then, slides were incubated for 30 min in blocking buffer containing 0.01 M PBS (pH 7.4), 10% normal goat serum (NGS), 0.05% thimerosal, 5% bovine serum albumin (BSA) and 0.3% Triton X-100. Subsequently sections were incubated overnight at 4° C. with peptide-affinity purified rabbit polyclonal antibody to GRM7 (inigenex, San Diego, Calif., USA), at a dilution of 1:150 in PBS with 0.3% Triton X-100 (tx-PBS). After extensive rinsing, biotinylated Fab fragments of goat anti-rabbit immunoglobulin (Ig) G (Rockland, Gilbertsville, Pa., USA), diluted 1:500 in tx-PBS, were added for 2 hours. Sections were washed and incubated for 2 hours in Cy3-conjugated streptavidin (Jackson Immunoresearch Laboratories, West Grove, Pa., USA), diluted 1:5000 in PBS. Finally, slides were rinsed in PBS, mounted with Citifluor (Ted Pella, Redding, Calif., USA) and studied with fluorescence and confocal microscopy. Human Immunohistochemistry

Human temporal bone sections were embedded in celloidin and stored in 80% ethanol. The sections were washed in 95% and 100% ethanol for 5 minutes followed by a wash in 100% methanol for 5 minutes. A non-heated antigen retrieval reagent (Saturated NaOH (Sigma)) and methanol at a concentration of 1:3 were applied to the sections. Sections were incubated in antigen retrieval reagent for 5 minutes, washed in 100% methanol for 5 minutes followed by a 5 minute wash in 80% methanol. The sections were rinsed in PBS for 10 minutes two times to remove the methanol. Each section was placed in Triton-X 100 (Sigma Cat. # X-100) for 10 minutes and washed in PBS 3 times for 10 minutes. The sections were incubated in 10% BSA (Sigma Cat. # A-2153) at room temperature for 30 minutes to block non-specific binding. After blocking, sections were incubated with the primary antibody (mGluR7 Imgenex Cat. #IMG-71406) used at a concentration of 2 μl/ml and left at 4° C. overnight. The following day sections were washed in PBS and incubated in a biotinylated secondary antibody (LSAB2 system AP: Mouse/rabbit, Dako Cat. # K0674) for 1 hr. After removal from incubation the sections were washed in PBS three times for 10 minutes and incubated in a tertiary antibody or label antibody—Streptavidin conjugated enzyme (LSAB2 system AP: Mouse/rabbit, Dako Cat. # K0674) for 1 hour. Following this incubation the sections were washed in PBS three times for 10 minutes and a few drops of fast red chromogen (Chromogen and Substrate Kit from Biogenex, Cat. #HK182-5K) were applied for about 3 to 5 seconds. The sections were washed in PBS to stop the chromogen reaction and placed in distilled water. Each section was then counterstained with Mayer's Hematoxylin (Biogenex, Cat. #HK100-5K) for 3 to 5 seconds, rinsed with tap water and ammonium water until the sections turned blue. Finally, each section was rinsed in distillied water and mounted onto a glass slide with aqueous mounting media (from Biogenex Cat. #HK099-5K) and cover slipped.

### Results

### GRM7 Expression in the Mouse and Human Inner Ear

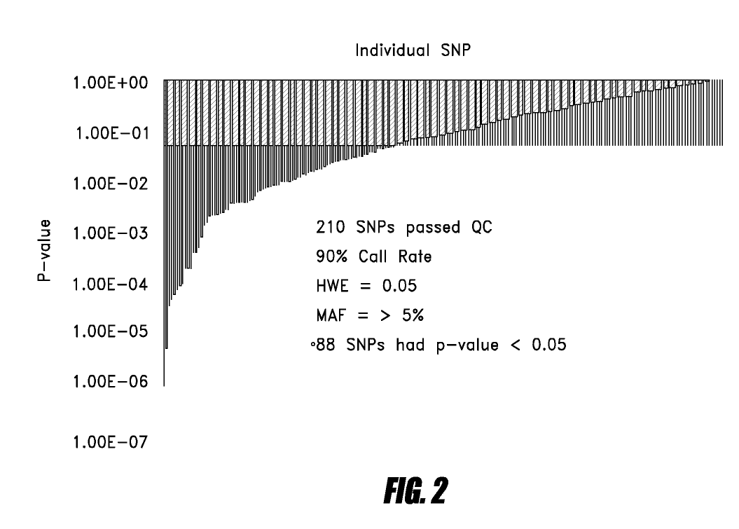

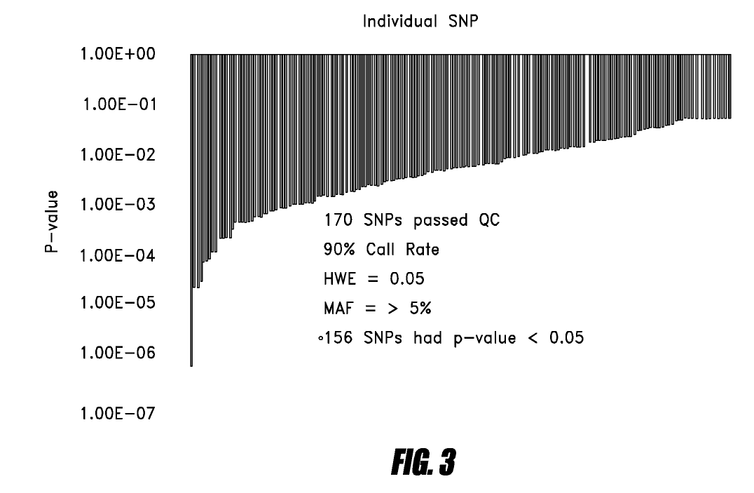

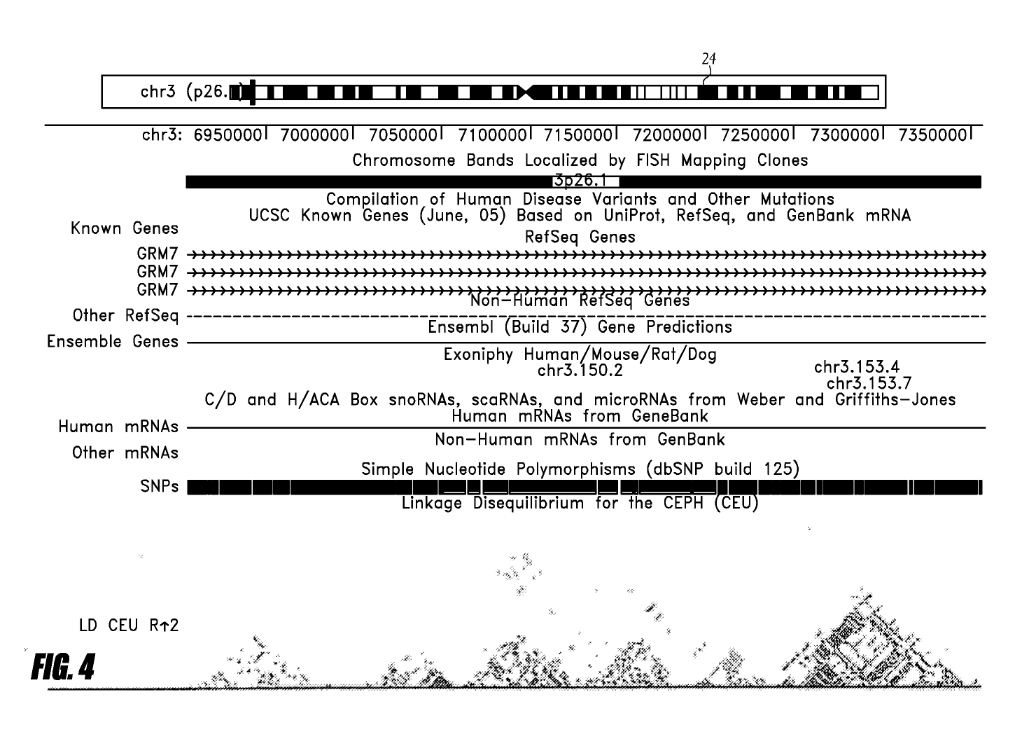

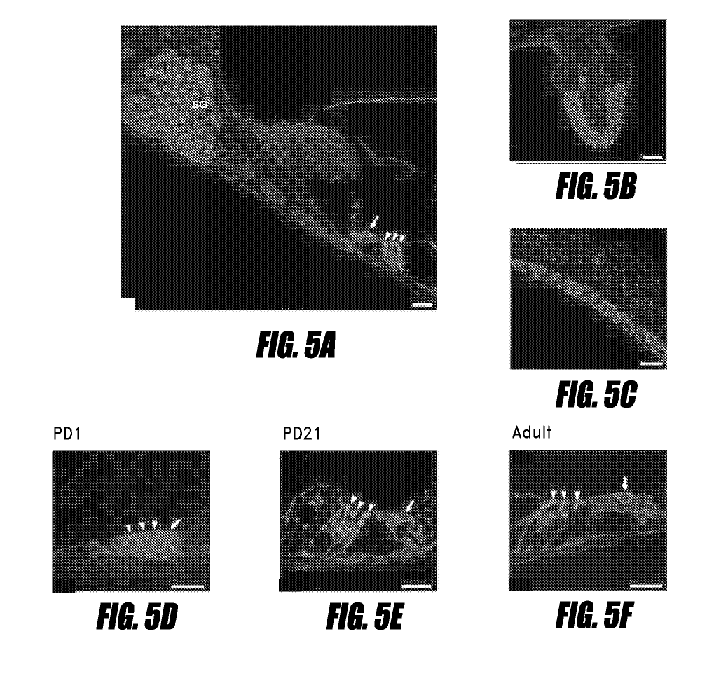

GRM7 expression was studied by immunohistochemistry in three different stages of development of the mouse inner ear (PD1, PD21 and adult; FIG. 5). Negative controls without primary antibody and/or without secondary antibody showed no staining. In every one of these developmental stages, GRM7 expression was concentrated in the neurons of the spiral ganglion (FIG. 5a), in the inner hair cell and outer hair cells of the organ of Corti (FIG. 5a, d, e, f) and the hair cells of the vestibular apparatus: the sacculus (FIG. 2c), the utriculus and the cristae ampullaris (FIG. 2b). At PD1, GRM7 labeling was not as bright as in the older stages, but still, a very clear specific signal was detected in the sensory epithelium of the organ of Corti (FIG. 5d), in the hair cells of the vestibular apparatus and in the spiral ganglion. At PD21 and in adult inner ears, expression of GRM7 was more abundant. There were no differences observed in staining intensity or staining pattern between basal and apical turns of the cochlea.

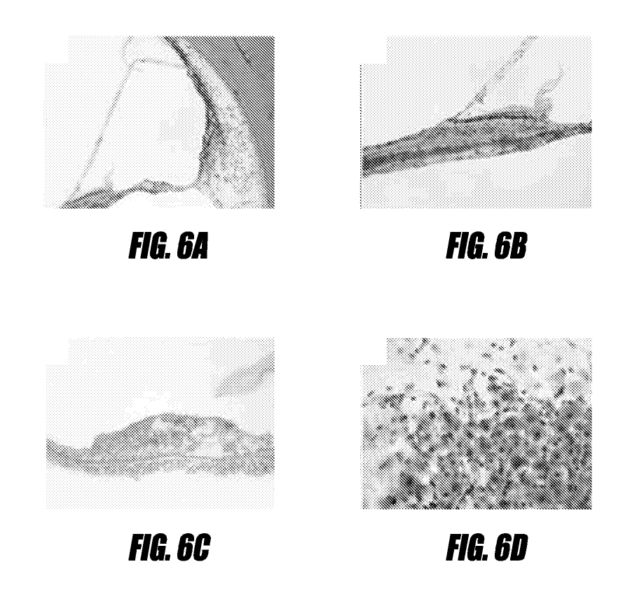

GRM7 expression was also studied in celloidin embedded adult human temporal bone specimens using immunohistochemistry. Negative controls without primary antibody and/or without secondary antibody showed no staining. GRM7 was detected in the interdental cells of the spiral limbus, the inner and outer hair cells and Hensens' cells of the organ of Corti and the type II fibrocytes of the spiral ligament (FIG. 6a, b, c). As in the mouse, GRM7 was also detected in the spiral ganglion neurons (FIG. 6d).

### Example 3

A 65 year old man presents with symptoms of presbycusis, including moderate loss of higher frequency (above 2000 Hz) hearing (50 decibel threshold) in both ears. The man is administered two drops per ear twice daily of a topical otic formulation comprising 10 ug/ml of AMN082 in a phosphate-buffered saline and further comprising 0.25% the carboxyvinyl water swellable gelling agent, Carbopol 934P; i.e., about 1 ug nGluR7 agonist per ear per dosing. After 14 days, the patient is tested by an audiologist. The threshold is lowered to 35 decibels right ear and 40 decibels left ear.

### Example 4

A 72 year old woman presents with symptoms of presbycusis, including loss of moderately high frequency (above 1000 Hz) hearing (40 decibel threshold right ear and 35 decibel threshold left ear). The woman is administered an oral formulation 4 times per day comprising 2 ug per dose of AMN082. The extended release capsule comprises a matrix of 10% by weight hydroxypropyl methylcellulose (e.g., Methocel®; Dow Chemical Company, U.S.A), and 50% by weight of a cornstarch filler. After 14 days, the threshold is lowered to 35 decibels right ear and 25 decibels left ear.

### Example 5

An 82 year old man presents with symptoms of presbycusis, including loss of hearing in the 500 to above 2000 Hz range. The man is implanted with an Alset® osmotic pump adapted for cochlear delivery; see e.g., Richardson, R. T., Noushi, F., O'Leary, S. Inner ear therapy for neural preservation. AUDIOLOGY AND NEURO-OTOLOGY 2006; 11(6): 343-356. The pump is supplied with an aqueous sterile solution comprising AMN082. The pump delivers a dose of 10 ug/hour of the mGluR7 modulator. After 10 days, hearing is improved in both ears.

What is claimed is:

1. Use of an mGluR7 modulator in the preparation of a medicament for the treatment or prevention of age-related hearing loss (presbicusis).

2. The use claim 1, wherein the modulator is a selective mGluR7 agonist.

3. The use of claim 2, wherein the selective mGluR7 agonist is AMN082.

4. The use of claim 1, wherein the medicament is formulated for oral, topical or osmotic pump delivery.

5. A method for treating or preventing age-related hearing loss (presbicusis), comprising administering to a mammal in need thereof an amount of a modulator of mGluR7 sufficient to treat and/or prevent said presbicusis.

6. The method of claim 1, wherein said modulator is a selective mGluR7 agonist.

7. The method of claim 2, wherein said selective mGluR7 agonist is AMN082.

8. A pharmaceutical composition for treating or preventing age-related hearing loss, comprising a modulator of mGluR7.
